(19) United States
(12) Patent Application Publication
(10) Pub. No.: US 2025/0213439 A1
(43) Pub. Date: Jul. 3, 2025

(54) Oral Care Compositions, Methods, and Kits

(71) Applicant: Solventum Intellectual Properties Company, Maplewood, MN (US)

(72) Inventors: Yizhong Wang, Woodbury, MN (US); Bradley D. Craig, Lake Elmo, MN (US)

(21) Appl. No.: 18/850,142

(22) PCT Filed: Mar. 7, 2023
(86) PCT No.: PCT/IB2023/052163

§ 371 Date: Sep. 24, 2024

Related U.S. Application Data
(60) Provisional application No. 63/362,482, filed on Apr. 5, 2022.

(51) Int. Cl. A61K 8/19; A61K 8/21; A61K 8/365; A61Q 11/00

(52) U.S. Cl. CPC A61K 8/19; A61K 8/21; A61K 8/365; A61Q 11/00

(57) ABSTRACT

An aqueous oral care composition includes calcium, fluoride, citrate, and water. The composition includes at least 2 moles of citrate for every 3 moles of calcium, and at least 1 mole of fluoride for every 1 mole of calcium. Water is present in the composition in an amount of at least 50 wt %, based on the total weight of the composition. The composition is a solution.

### BACKGROUND

Oral care compositions having fluoride and calcium ions have been described in, for example, U.S. Pat. Nos. 10,064,802 and 10,682,300.

### DETAILED DESCRIPTION

Fluoride has been a staple material in monograph mouth rinses, toothpastes, varnishes, and the like for many decades. Recently, attempts have been made to incorporate additional calcium or phosphate ions to aid in the remineralization of teeth and the formation of hydroxyapatite and fluoroapatite within the tooth structure. The limitations encountered with conventional materials are several-fold. First, trying to put calcium ions and fluoride ions into the same aqueous solutions leads to the formation of highly insoluble calcium fluoride, which has a solubility in water of only 8-10 ppm and which will normally precipitate out of solution. This limits the available calcium and fluoride that one can put into the aqueous composition to try and achieve therapeutic benefit. Second, the addition of compounds like calcium phosphate or even particles of hydroxyapatite have been tried in various formulations in the past, but the issue surrounding these types of materials is that in order to release the calcium or phosphate within them, it requires the same physiological pH that is required to dissolve the hydroxyapatite of the tooth structure, which is counterproductive. Third, the addition of other desirable ions, such as zinc or strontium also leads to insoluble fluorides with minimal ionic bioavailability. Consequently, formulations containing fluoride and calcium and/or other beneficial cations in a single solution with more bioavailable ions are desirable as such compositions are beneficial for the uptake of those ions in tooth structure and provide additional benefits (e.g., antimicrobial activity, matrix reinforcement of the tooth, and the like).

The term “metastable” refers to a quasi-equilibrium state of a material in which the amount of free energy is greater than that contained in the equilibrium state.

The term “supersaturated” refers to a composition (e.g., an aqueous solution) that contains more solute (e.g., calcium citrate or its hydrates) therein than is soluble at equilibrium conditions.

The term “chemical complex” refers to a molecular entity formed by a loose bond containing two or more constituent molecular entities (ionic or uncharged), or the corresponding chemical species. The bond between components of the complex is usually weaker than a covalent bond. Thus, the term “chemical complex” can be considered any combination of components in which the molecules of each component are mixed and weakly bound to each other. The term “chemical complex” does not necessarily have to be an ionic or other bond between components. It also does not include covalent bonds between the components of the complex.

The term “solution” refers to a homogeneous mixture of two or more substances (a solvent and one or more solutes) in relative amounts that can be varied continuously up to the limit of solubility of any one of the solutes in the solvent (under standard temperature and pressure conditions). As used herein, a “solution” has no amount of solute (e.g., in the form of precipitate) that is visible to the unaided human eye, but may include nanoparticles (e.g., particles having an average longest dimension of less than 50 nanometers, less than 20 nanometers, or less than 10 nanometers) that are not visible to the unaided human eye.

The term “aqueous solution” refers to a solution in which water is the solvent.

The term “free fluoride” refers to isolated fluoride in a solution (e.g., the amount of fluoride in a solution that is not formed up into an insoluble complex or particle), and its concentration in the solution can be determined by a meter with a fluoride-selective electrode (a fluoride probe).

The term “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

In this application, terms such as “a,” “an,” and “the” are not intended to refer to only a singular entity, but include the general class of which a specific example may be used for illustration. The terms “a,” “an,” and “the” are used interchangeably with the phrases “at least one” and “one or more.” The phrases “at least one of” and “comprises at least one of” followed by a list refers to any one of the items in the list and any combination of two or more items in the list.

The term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about”. As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

In some embodiments, the present disclosure relates to methods of stabilizing calcium and other ions useful in oral care solutions while in the presence of fluoride. This provides the potential to deliver ions for remineralization, anti-microbial effect, and other potential utility in dental and/or orthodontic compositions. Generally, the methods of the present disclosure employ a particular order and timing of material combination, as well as the chelating effect of citric acid (a trifunctional carboxylic acid) or, in some embodiments, etidronic acid (a bis-phosphonic structure). Surprisingly, it has been discovered that the order and timing of addition of certain components is critical to achieving a stable solution in a pH regime that is acceptable for oral applications.

In some embodiments, the method may include reacting (in aqueous solution) a calcium source with a chelating agent (e.g., etidronic acid or citric acid) to form a metastable (e.g., supersaturated metastable) aqueous solution of calcium citrate, calcium etidronate or their hydrates. The method may then include, soon after formation of the metastable solution, adding a water-soluble fluoride salt or salt solution to the metastable solution. Specifically, in some embodiments, the water-soluble fluoride salt or salt solution may be added to the metastable supersaturated solution prior to precipitation of the calcium citrate or its hydrates. In some embodiments, the water-soluble fluoride salt or salt solution may be added to the metastable supersaturated solution within 0.5 minutes, within 1.0 minutes, within 1.5 minutes, within 2.0 minutes, within 3.0 minutes, or within 5.0 minutes of formation of the supersaturated metastable solution. Surprisingly, it was discovered that if the fluoride salt solution is added within this time frame, an aqueous solution with a complex of citrate, calcium, and monovalent ions can be formed. Such aqueous solutions were observed to be clear, without visible precipitate, and with up to 1000's of ppm of measurable free fluoride in the solution. If the fluoride salt solution is not added in this timeframe, however, precipitation of insoluble calcium citrate occurs and no complex is formed (i.e., the fluoride must be added prior to the precipitation of the supersaturated calcium citrate or its hydrates).

In some embodiments, the metastable solution (e.g., metastable supersaturated solution) may include water and the reaction product of a calcium source and a chelating agent.

In some embodiments, suitable calcium sources for the metastable solution may include calcium hydroxide, calcium acetate, calcium carbonate, calcium hydrogen carbonate, or a salt such as the hydrate of calcium nitrate. In some embodiments, suitable calcium sources may include calcium hydroxide or calcium acetate.

In some embodiments, the chelating agent may include citric acid or etidronic acid. It has been discovered that citric acid is stable in higher concentrations than its etidronate counterparts. Consequently, in some embodiments, the chelating agent may include citric acid. It some embodiments, citric acid may be included as a mixture of carboxylic acid or phosphonic with citric acid. It was discovered that the calcium ions of the aqueous solutions need at least 2 moles of chelating acid groups per mole of calcium ion to form a stable solution, and consequently citric acid's three moles of chelating functional group (carboxylic acid or carboxylate) are sufficient. Additional chelating groups (e.g., via a mixture of citric acid with additional acids or by adding additional citric acid), but not less, may be employed without impacting the stability.

In some embodiments, the calcium source may be present in the metastable solution (e.g., metastable supersaturated solution) in an amount of at least 0.0005 wt % (or 5 parts per million (by weight) (ppm)), at least 0.005 wt % (or 50 ppm), at least 0.05 wt % (or 500 ppm), at least 0.5 wt %, or at least 5.0 wt %, based on the total weight of the solution; and the chelating agent may be present in the metastable solution in an amount such that the mole ratio of calcium ions to chelating functional acidic groups is at least 1 to 2; and the mole ratio of calcium ions to citric acid is no more than 1.5:1.

In some embodiments, water may be present in the metastable solution (e.g., metastable supersaturated solution) in an amount of at least 50 wt %, at least 70 wt %, at least 80 wt %, at least 90 wt %, or at least 99 wt %, based on the total weight of the solution.

In some embodiments, the water-soluble fluoride solution may include a fluoride salt and water. Suitable fluoride salts may include sodium fluoride, potassium fluoride, amine fluoride, silver diamine fluoride, or ammonium fluoride. In some embodiments, the fluoride salts may include sodium fluoride or ammonium fluoride. It was discovered that with sodium and ammonium fluoride, a higher concentration of free fluoride and greater stability could be achieved than with potassium fluoride.

In some embodiments, the fluoride salt may be present in the water-soluble fluoride solution at a concentration of at least 0.00024 wt % (or 2.4 ppm), at least 0.0024 wt % (or 24 ppm), at least 0.024 wt % (or 240 ppm), at least 0.24 wt % (or 2400 ppm), at least 2.4 wt %, or at least 5 wt %, based on the total weight of the water-soluble fluoride solution; and water may be present in the water-soluble fluoride solution in amount of at least 50 wt %, at least 70 wt %, at least 80 wt %, at least 90 wt %, or at least 99 wt %, based on the total weight of the water-soluble fluoride solution.

As previously discussed, the methods of the present disclosure may produce an aqueous solution that includes calcium, fluoride, and citrate. In some embodiments, the aqueous solution may include free fluoride, a complex including any or all of calcium and citrate, and fluoride, and stable calcium fluoride (CaF₂) nanoparticles dispersed in the solution. Such solution may be considered a one-part composition. The CaF₂ nanoparticles may be stable. That is, that they remain suspended or dissolved in the solution such that the nanoparticles do not (or do not substantially) grow or precipitate out of solution.

In some embodiments, free fluoride may be present in the aqueous solution an amount of at least 0.0002 wt % (or 2.0 ppm), at least 0.00024 wt % (or 2.4 ppm), at least 0.001 wt % (or 10 ppm), at least 0.002 wt % (or 20 ppm), at least 0.01 wt % (or 100 ppm), at least 0.05 wt %, or at least 0.50 wt %, based on the total weight of the aqueous solution. In some embodiments, free fluoride may be present in an amount of up to 1.0 wt %, up to 2.0 wt %, up to 5.0 wt %, or up to 10 wt %, based on the total weight of the aqueous solution.

In some embodiments, the calcium may be present in an amount of at least 0.0005 wt % (or 5 ppm), at least 0.002 wt % (or 20 ppm), at least 0.02 wt % (or 200 ppm), at least 0.2 wt % (or 2,000 ppm), or at least 0.5 wt %, based on the total weight of the aqueous solution. In some embodiments, the calcium may be present in an amount of up to 1.0 wt %, up to 2.0 wt %, up to 5.0 wt %, or up to 10 wt %, based on the total weight of the aqueous solution.

In some embodiments, the molar ratio of citrate to calcium in the aqueous solution may be such that there are at least 2 moles of citrate for every 3 moles of calcium. It was discovered that if the molar ratio of citrate to calcium ions in the aqueous solution is less than this, precipitation occurs.

In some embodiments, the molar ratio of fluoride to calcium in the aqueous solution may be such that there is at least 1 mole of fluoride for every 1 mole of calcium. It was discovered that if the molar ratio of fluoride to calcium is less than this, precipitation occurs.

In some embodiments, the aqueous solution may include water as a solvent to form a clear solution. In some embodiments, water may be present in the aqueous solution an amount such that it occupies 100 wt %, at least 90 wt %, at least 80 wt %, or at least 70 wt % of the weight of the aqueous solution not occupied by calcium, fluoride, and citrate.

In some embodiments, the aqueous solution may include calcium fluoride nanoparticles dispersed therein. The nanoparticles may have an average longest dimension of less than 50 nanometers (nm), less than 20 nm, less than 10 nm, or less than 5 nm. In some embodiments, the calcium fluoride nanoparticles may be present in the aqueous solution at a concentration and size such that the nanoparticles are not visible in the aqueous solution (to the unaided human eye)

In some embodiments, after the aqueous solution aqueous is prepared to form a stable solution, additional fluoride may be added to achieve higher concentration of free fluoride in the solution. In this regard, additional fluoride may be provided such that at least an additional 0.1 wt %, an additional 0.5 wt %, an additional 1.0 wt %, an additional 2.0 wt %, or an additional 5 wt % of fluoride is present, based on the total weight of the stable aqueous solution upon formation up to the solubility limit of the fluoride salt.

As previously discussed, in some embodiments, the oral care one-part compositions (e.g., solutions) of the present disclosure are aqueous compositions (e.g., solutions), although they may include a small amount of one or more organic solvents. Examples of organic solvents are selected from ethanol, isopropanol, dimethyl sulfoxide (DMSO), isoprene sulfone (IS), butadiene sulfone (BS), piperylene sulfone (PS), ethyl acetate, methyl acetate, isopropyl acetate, acetone, methyl ethyl ketone (MEK), methyl isobutyl ketone (MIBK), and combinations thereof.

In some embodiments, the aqueous oral care one-part compositions (e.g., solutions) are free of organic solvents that function as liquid carriers (as opposed to organic solvents that are used as carriers/solvents for flavorants or sweeteners). For example, certain additives may be provided as a solution or dispersion in an organic solvent as a liquid carrier. If there is any organic solvent (that functions as a liquid carrier) present in aqueous oral care one-part compositions (e.g., solutions) of the present disclosure, it is present in an amount of less than 5 wt-%, based on the total weight of the aqueous composition (e.g., solution).

In some embodiments, aqueous oral care compositions of the present disclosure are solutions that, when stored at room temperature and atmospheric pressure in a sealed container, are shelf stable for at least 2 months, at least 3 months, at least 4 months, at least 5 months, or at least 6 months without precipitation (detectable to the unaided human eye). Thus, aqueous oral care solutions of the present disclosure may be stored and remained clear (i.e., transparent or translucent without any cloudiness) for at least 2 months, at least 3 months, at least 4 months, at least 5 months, or at least 6 months.

As previously mentioned, the aqueous oral care compositions of the present disclosure may exhibit the above-described stability while also having a pH that that is acceptable for oral applications. In this regard, the aqueous oral care compositions may have a pH of between 2 and 11 or 3 and 10.

In some embodiments, the aqueous oral care one-part compositions (e.g., solutions) of the present disclosure can include a pharmaceutically acceptable buffer. The type and amount of such buffer may be selected to provide an oral care composition (e.g., solution) with a pH of at least 3.0, at least 3.5, at least, 4.0, at least 4.5, at least 5.0, at least 5.5, at least 6.0, or at least 6.5 In certain embodiments, the type and amount of such buffer may be selected to provide an oral care composition (e.g., solution) with a pH of up to 10, up to 9, up to 8.5, up to 7.5, or up to 7. In certain embodiments, the type and amount of such buffer may be selected to provide an oral care composition (e.g., solution) with a pH of 6.5 to 7.5, or a pH of 7.0. A wide variety of suitable pharmaceutically acceptable buffers can be included. Examples include acetate (e.g., sodium acetate), sodium carbonate, citrate (e.g., sodium citrate), tartrate, glycylglycine, histidine, glycine, lysine, arginine, glycine, sodium dihydrogen phosphate, disodium hydrogen phosphate, sodium phosphate, tris (hydroxymethyl)-aminomethane, potassium phosphate, potassium hydrogen phosphate, potassium dihydrogen phosphate, or mixtures thereof.

Aqueous oral care one-part compositions (e.g., solutions) of the present disclosure can also contain one or more active agents in addition to a source of fluoride. When included, the one or more additional active agents usually, but not always, include one or more active agents that are active in the oral cavity against disorders, diseases, or conditions of the teeth, gums, cheeks, tongue, roof of the mouth, and the like.

In some embodiments, the solutions of the present disclosure may further include phosphate. The phosphate may be present in the form of a water soluble phosphate salt such as ammonium phosphate, sodium dihydrogen phosphate, disodium hydrogen phosphate, sodium phosphate, tris (hydroxymethyl)-aminomethane, potassium phosphate, potassium hydrogen phosphate, potassium dihydrogen phosphate, or mixtures thereof.

In order to form fluorapatite, in addition to calcium and fluoride, phosphate need be present. While low levels of phosphate may be present in saliva, it is desirable to have an additional phosphate source. It was discovered that phosphates could be added to the compositions of the present disclosure without undoing the stabilization benefits discussed above.

Examples of additional active agents that can be employed include one or more whitening agents, anticalculus agents, remineralization agents, stannous sources, antimicrobial agents, antioxidants, saliva stimulating agents, breath freshening agents, antiplaque agents, anti-inflammatory agents, H₂ antagonists, desensitizing agents, nutrients, and proteins. Various combinations of such additional active agents may be used if desired. When employed, one or more additional active agents will be typically used in amounts sufficient to achieve their intended effect.

When employed, the antimicrobial agents can include a wide variety of orally acceptable antimicrobial agents. Examples include triclosan, 8-hydroxyquinoline, zinc ion, stannous ion, cupric compounds, phthalic acid and salts thereof, quaternary ammonium compounds, sanguinarine, salicylanilide, salicylic acid, thymol, eugenol, neomycin, kanamycin, clindamycin, amoxicillin, tetracycline, doxycycline, minocycline, metronidazole, chlorohexidine, and the like.

When employed, the antioxidants can be a wide variety of orally acceptable antioxidants. Examples include butylated hydroxy anisone, butylated hydroxy toluene, vitamin A, carotenoids, vitamin E, flavonoids, polyphenols, ascorbic acid or salts thereof, chlorophyll, melatonin, and the like.

When employed, the saliva stimulants can be a wide variety of orally acceptable saliva stimulants. Examples include lactic acid, succinic acid, ascorbic acid, adipic acid, fumaric acid, and tartaric acid.

When employed, the breath freshening agents can be a wide variety of orally acceptable breath freshening agents. Examples include zinc salts such as zinc salts of gluconate, citrate, chlorite, alpha-ionone, and the like.

When employed, the antiplaque agents can be a wide variety of orally acceptable antiplaque agents. Examples include stannous salts, salts of copper, magnesium or strontium, dimethicone copolyols, such as cetyl dimethicone copolyol, papain, glucamylase, glucose oxidase, urea, calcium lactate, calcium glycerophosphate, strontium polyacrylates, and the like. Further examples of antiplaque agents include biofilm inhibition agents, particularly those described in U.S. Pat. No. 8,968,709 (Yang et al.).

When employed, the anti-inflammatory agents can be a wide variety of orally acceptable anti-inflammatory agents. Examples include steroids such as flucinolone and hydrocortisone, non-steroidal anti-inflammatory drugs such as ketorolac, flurbiprofen, ibuprofen, naproxen, indomethacin, diclofenac, etodolac, indomethacin, sulindac, tomlmetin, ketoprofen, fenoprofen, piroxicam, nabumetone, acetyl salicylic acid, salicylic acid, diflunisal, meclofenamate, mefenamic acid, oxyphenbutazone, phenylbutazone, and the like.

When employed, the H₂ antagonists can be a wide variety of orally acceptable H₂ antagonists. Examples include cimetidine, etinidine, ranitidine, tiotidine, lupitidine, denetidine, famotidine, roxatidine, pifatidine, lamtidine, zaltidine, nizatidine, mifentidine, ramixotidine, loxtidine, bisfentidine, sufotidine, ebrotidine, impromdine, and the like.

When employed, the desensitizing agents can be a wide variety of orally acceptable desensitizing agents. Examples include potassium citrate, potassium chloride, potassium tartrate, potassium bicarbonate, potassium oxalate, potassium nitrate, strontium salts, arginine, acetyl salicylic acid or salts thereof, salicylic acid or salts thereof, codeine, acetaminophen, and the like.

In some embodiments, any number of additional conventional orally acceptable remineralization agents may be employed.

When employed, the nutrients can be a wide variety of orally acceptable nutrients. Examples include vitamins, such as vitamins C, D, thiamine, riboflavin, folic acid, nicotinamide, niacin, pyridoxine, bioflavonoids, and the like, supplements, such as amino acids, lipotropics, fish oil, polyunsaturated fatty acids, eicosapentanoic acid, docosahexanic acid, coenzyme Q10, ubiquinone, minerals such as potassium, and the like.

When employed, the proteins can include a wide variety of orally acceptable proteins. Examples include milk proteins, peroxide producing enzymes, amylase, papain, glucoamylase, glucose oxidase, and the like.

In certain embodiments, aqueous oral care one-part compositions (e.g., solutions) of the present disclosure may optionally include a thickener to provide a composition (e.g., solution) with a suitable viscosity to allow for the desired method of application. For example, a suitable thickener in a sufficient amount may be used to achieve a composition (e.g., solution) viscosity adequate to maintain the composition (e.g., solution) in an inverted mouthpiece tray applicator for up to four minutes (typical time for a professionally applied fluoride treatment), and yet be fluid enough to have acceptable handling characteristics for the dental operator (e.g., when dispensing into a dental tray applicator). Or, a suitable thickener in a sufficient amount may be used to achieve a viscosity adequate to paint on a tooth surface. Or, a suitable thickener in a sufficient amount may be used to achieve a viscosity adequate to stick to the tooth surface as a thick gel/gum/strip.

In certain embodiments, the type and amount of thickener may be selected to provide an oral care composition (e.g., solution) with a viscosity of at least 0.05 Pascal seconds at a shear rate of 1.0/second. In certain embodiments, a type and amount of thickener is selected to provide an oral care composition (e.g., solution) with a viscosity of up to 0.5 Pascal seconds at a shear rate of 1.0/second. In certain embodiments, a type and amount of thickener is selected to provide an oral care composition (e.g., solution) with a viscosity of up to 500 Pascal seconds or up to 5000 Pascal seconds, at a shear rate of 1.0/second.

In certain embodiments, a thickener may be present in an oral care one-part composition (e.g., solution) in an amount of between 0.1 wt-% and 10 wt-% or between 0.1 wt-% and 5 wt-%. In certain embodiments, a thickener is present in an oral care one-part composition (e.g., solution) in an amount of less than 2.5 wt-%, based on the total weight of the aqueous composition (e.g., solution). In certain embodiments, a thickener is present in an amount of at least 0.5 wt-%, based on the total weight of the aqueous composition (e.g., solution).

Suitable thickeners are typically those that are generally safe for human ingestion (FDA approved for internal use), do not bind fluoride ions, and do not significantly affect the bioavailability of fluoride ions.

In certain embodiments, the thickener is selected from natural gums, water soluble cellulose derivatives (such as hydroxyethyl cellulose, carboxylic methyl cellulose), inorganic fillers (e.g., colloidal silica, fumed silica, alumina, titania, and zinc oxide), alkylene oxide polymers (e.g., polyethylene glycol, polypropylene glycol, and copolymers of polyethylene glycol and polypropylene glycol), modified water-soluble starches, crosslinker polyacrylic acids, and combinations thereof.

In certain embodiments, aqueous oral care compositions (e.g., solutions) of the present disclosure may include one or more optional additives including flavoring agents (i.e., flavorants) and sweeteners. Other optional additives include surfactants. Various combinations of such additives may be used if desired. Other optional additives include preservatives.

In certain embodiments, aqueous oral care compositions (e.g., solutions) of the present disclosure may include a surfactant. Typically, such surfactant is an anionic surfactant, examples of which include polysorbate, glycerol, polyglycerol-based surfactant, or combinations thereof. When present, a surfactant can be used in any suitable amount, most often in an amount sufficient to impart wettability. A suitable amount is typically 0.1 wt-% to 5.0 wt-%, based on the total weight of the aqueous composition (e.g., solution).

In certain embodiments, aqueous oral care compositions (e.g., solutions) of the present disclosure may include a preservative, such as potassium sorbate, CPC, CHG, methyl paraben, ethyl paraben, propyl paraben and any other paraben-based preservative and mixtures. A suitable amount is typically 0.001 wt-% to 5.0 wt-%, based on the total weight of the aqueous composition (e.g., solution).

In certain embodiments, aqueous oral care compositions (e.g., solutions) of the present disclosure are included in kits. Typically, such kit includes an applicator (e.g., dental brush, cotton tip swab) for the oral care composition (e.g., solution). Such applicator may be integrated into a container having the oral care composition (e.g., solution) therein.

In certain embodiments, the oral care composition (e.g., solution) is provided in individual sealed unit dose containers. In use, the seals of such individual sealed unit dose containers are broken and the composition (e.g., solution) picked up with the applicator and the composition (e.g., solution) applied to a tooth surface.

In certain embodiments, the oral care composition (e.g., solution) is provided in a multi-dose container. In use, a drop of the composition (e.g., solution) can be dispensed onto a tray, piece of plastic, piece of paper, dish, well, pan, etc., and the composition (e.g., solution) picked up with the applicator and the composition (e.g., solution) applied to a tooth surface.

In certain embodiments, the kit may further include one or more of a dental restorative, a tray, a dish, a well, or a pan. Examples of dental restorative include, but are not limited to, an adhesive, primer, cement, liner, sealant, amalgam, resin, resin composite, glass ionomer, resin-modified glass ionomer, glass-ceramic, ceramic, metal, plastic, or combination thereof.

An aqueous oral care composition (e.g., solution) of the present disclosure can be made using any techniques known to one of skill in the art. In certain embodiments, the components may be added together as described above and dissolved.

In certain embodiments, an aqueous oral care composition (e.g., solution) of the present disclosure is used in a method of providing fluoride to a patient's tooth surface. The method includes applying the aqueous oral care composition (e.g., solution) described herein to the patient's tooth surface. The aqueous oral care composition, alone, may be applied, or the aqueous oral care composition may be incorporated into another composition (e.g., a tooth paste, mouth rinse, varnish) before being applied.

The present disclosure also provides methods, such as providing fluoride and calcium to a patient's tooth surface, as well as reducing the incidence of dental caries, for example. Such methods involve applying an aqueous oral care one-part composition as described herein to the patient's tooth surface.

In certain embodiments, applying an aqueous oral care one-part composition (e.g., solution) includes painting the oral care one-part composition (e.g., solution) on the patient's tooth surface.

In certain embodiments, applying an aqueous oral care one-part composition (e.g., solution) includes dispensing the oral care one-part composition (e.g., solution) into a dental tray and attaching the tray having the oral care one-part composition (e.g., solution) therein to the patient's tooth surface. In certain embodiments, the dental tray includes an orthodontic aligner treatment tray.

In certain embodiments, an aqueous oral care composition (e.g., solution) of the present disclosure is used in a method of reducing the incidence of dental caries (e.g., by preventing or arresting dental caries) in a patient in need thereof. The method includes applying the aqueous oral care composition (e.g., solution) described herein to the patient's tooth surface.

In certain embodiments, an aqueous oral care composition (e.g., solution) of the present disclosure is used in a method of reducing dentin sensitivity and/or root sensitivity (e.g., during cavity treatment and/or on an exposed root) in a patient in need thereof. The method includes applying the aqueous oral care composition (e.g., solution) described herein to the patient's tooth surface.

In certain embodiments, an aqueous oral care composition (e.g., solution) of the present disclosure is used in a method of treating a patient's tooth surface. The method includes applying the aqueous oral care one-part composition (e.g., solution) disclosed herein to the patient's tooth surface to form a treated tooth surface, and optionally applying a dental restorative to the treated tooth surface.

In certain embodiments, a patient's tooth surface that is treated with a method as described herein includes enamel, dentin, cementum, root, or combinations thereof.

In certain embodiments of the methods described above, applying includes painting the oral care composition (e.g., solution) on the patient's tooth surface. In certain embodiments of the methods described above, applying includes dispensing the oral care composition (e.g., solution) into a dental tray (e.g., an orthodontic aligner treatment tray) and attaching the tray having the oral care composition (e.g., solution) therein to the patient's tooth surface.

In certain embodiments of the methods described above, the oral care composition (e.g., solution) is subsequently dried (e.g., using flowing air) after being applied to the tooth surface.

In certain embodiments of the methods described above, the oral care composition (e.g., solution) is subsequently wiped with cotton, paper, and any other wiping material to remove excess oral care composition (e.g., solution) on the tooth surface after being applied to the tooth surface.

In certain embodiments of the methods described above, the methods further include placing a dental restorative on the tooth surface having the oral care composition applied thereto. In certain embodiments of the methods described above, the methods further include combining the oral care composition and a dental restorative to form a dental restoration composition and then applying the dental restoration composition to a tooth. Examples of dental restorative include, but are not limited to, an adhesive (such as 3M SCOTCHBOND Universal Adhesive (available from 3M Company of St. Paul, MN, USA), primer, cement (such as 3M RelyX UNICEM 2 AUTOMIX Self-Adhesive Resin Cement, available from 3M Company of St. Paul, MN, USA), liner (such as 3M ESPE VITREBOND Plus Light Cure Glass Ionomer Liner/Base), sealant, amalgam, resin, resin composite (3M FILTEK Z250 Universal Restorative), glass ionomer (such as 3M KETAC Universal APLICAP Glass Ionomer Restorative), resin-modified glass ionomer (such as RelyX Luting Plus RMGI Cement), glass-ceramic, ceramic, metal, plastic, or combination thereof.

In certain embodiments of the methods described above, after drying the oral care composition on the tooth, the methods may further include coating over the dried oral care composition with a layer of a moisture-permeable polymer to, for example, allow moisture penetration for the treatment of incipient caries, white spots, or the like.

### EXAMPLES

TABLE 1
Materials List

| ABBREVIATION/ | | |
|---|---|---|
| NAME | DESCRIPTION | SOURCE |
| NH₄F | ammonium fluoride | Honeywell |
| Arginine | L-arginine | VWR |
| BHT | butylated hydroxytoluene | Sigma-Aldrich |
| Ca(OH)₂ | calcium hydroxide | VWR |
| Ca(NO₃)₂•4 H₂O | calcium nitrate tetrahydrate | VWR |
| Cellulose gum | sodium carboxymethylcellulose (CMC); | Ashland Chemical |
| | AQUALON CMC 7MF | |
| Citric acid | citric acid, anhydrous ACS | VWR |
| CPQ | Camphorquinone | Sigma-Aldrich |
| DPIHFP | Diphenyl Iodonium | Alfa Aesar |
| | Hexafluorophosphate | |
| EDMAB | Ethyl 4-(N,N-dimethylamino)benzoate | Sigma-Aldrich |
| EDMOA | 2-ethyl-9,10-dimethoxyanthracene | Sigma-Aldrich |
| Etidronic acid (60% | 1-Hydroxyethylidene-1,1-diphosphonic | Sigma-Aldrich |
| aqueous solution) | acid; HEDP; CAS No. 2809-21-4 | |
| FeCl₃•6H₂O | iron (III) chloride hexahydrate | VWR |
| Fumaric acid | fumaric acid (99%) | VWR |
| Fumed silica | AEROSIL R812S, hydrophobic fumed | Evonik Industries |
| | silica | |
| GDMA | glycerol dimethacrylate | Sigma-Aldrich |
| CDMA | The reaction product between citric acid | 3M prepared internally |
| | and 2-isocyanatoethyl methacrylate as | |
| | described in Preparatory Example 2 of | |
| | U.S. Pat. No. 5,922,786 (Mitra et al.) | |
| CDMA/GDMA | Mixture (50/50) of GDMA and | 3M prepared internally |
| | CDMA | |
| Glycerin | glycerin (USP, food grade) | Cargill |
| HEMA | 2-hydroxyethyl methacrylate | Sigma-Aldrich |
| HEC250 | hydroxyethyl cellulose; HEC250 pharm | Ashland |
| | M grade | |
| KNO₃ | potassium nitrate | VWR |
| KH₂PO₄ | potassium dihydrogen phosphate | Spectrum Chemical |
| KF | potassium fluoride | Spectrum Chemical |
| K₂S₂O₈ | potassium persulfate | Sigma-Aldrich |
| K₂SO₄ | potassium sulfate | Spectrum Chemical |
| K sorbate | potassium sorbate, powder, FCC | Spectrum Chemical |
| maleic acid | maleic acid, FCC | Spectrum Chemical |
| RelyX ™ Luting Plus | RelyX ™ Luting Plus Resin Modified | 3M Company |
| RMGI | Glass Ionomer Cement | |
| ZEODENT 165 | ZEODENT 165 hydrated silica | Evonik Silica |
| SIDENT 9 | SIDENT 9 abrasive silica | Evonik Industries |
| silica/zirconia filler | Silane-treated zirconia-silica (Zr—Si) | 3M prepared internally |
| | filler was prepared as described in U.S. | |
| | Pat. No. 4,503,169 (Randklev). | |
| Na citrate | sodium citrate, anhydrous, FCC | Spectrum Chemical |
| Na₂HPO₄ | sodium hydrogen phosphate | Spectrum Chemical |
| sodium lauryl sulfate | sodium lauryl sulfate | TCI |
| Na₃PO₄ | sodium phosphate | Spectrum Chemical |
| sodium saccharin | sodium saccharin | Spectrum Chemical |
| sodium benzoate | sodium benzoate, powder, FCC | Spectrum Chemical |
| NaF | sodium fluoride | Spectrum Chemical |
| Sorbitol | sorbitol, powder, FCC | Spectrum Chemical |
| spearmint flavor | spearmint flavor | International Flavor & |
| | | Fragrances Inc. |
| Sucralose | sucralose, FCC, 98-102% | Spectrum Chemical |
| SR454 | Ethoxylated (3) trimethylolpropane | Sartomer Americas |
| | triacrylate | |
| Tartaric acid | L-tartaric acid | VWR |
| TiO₂ | Titanium oxide | DUPONT |
| VBCP | Reaction product of 2 - isocyanatoethyl | 3M prepared internally |
| | methacrylate and a copolymer of acrylic | |
| | acid and itaconic acid prepared as | |
| | described in U.S. Pat. No. 5,130,347, | |
| | Example 11. | |

### Example Preparation Procedure

The general procedure for preparing the examples was to combine measured ingredients according to the tables and description below in the following order. First the calcium compound was dissolved in water, followed by the addition of citric acid (or other chelating agent, e.g., etidronic acid), the mixture was mixed well to form a solution. Next the fluoride containing compound was added and mixed well. For some examples an alternative, yet equivalent procedure was used: an aqueous solution of the calcium compound and citric acid (or alternative chelating agent) was prepared separately and then mixed with a second aqueous solution of the fluoride containing compound to achieve the desired concentrations of ingredients. The key order of addition is to first prepare the metastable aqueous solution comprising calcium citrate or its hydrates (or adequate chelating agent) and then add the fluoride containing compound, typically within 5 minutes. The example solutions were observed for at least 24 hours to ensure they remained a solution and no precipitate was formed. Working examples of the invention were stable and formed no precipitate. Comparative examples were not stable solutions in that they formed a visible precipitate, sometimes immediately, or at least within 24 hours of preparation.

TABLE 2
Examples EX-1A-EX-1E, and Comparative Example CEx. A

| Examples with NH₄F | EX-1A | EX-1B | EX-1C | EX-1D | EX-1E | CEx. A |
|---|---|---|---|---|---|---|
| Components | wt/wt % | wt/wt % | wt/wt % | wt/wt % | wt/wt % | wt/wt % |
| Ca(OH)₂ | 1.58 | 0.79 | 0.72 | 0.71 | 0.79 | 0.79 |
| Citric acid | 4.12 | 3.00 | 1.45 | 2.12 | 2.06 | 2.06 |
| NH₄F | 1.58 | 0.79 | 0.72 | 0.71 | 0.790 | 0.79 |
| fumaric acid | — | — | 0.48 | — | — | — |
| water | 92.72 | 95.42 | 96.64 | 96.45 | 96.36 | 96.36 |
| total | 100.0 | 100.0 | 100.0 | 100.0 | 100 | 100.0 |
| moles Ca(OH)₂ | 0.0213 | 0.0107 | 0.0097 | 0.0096 | 0.0107 | 0.0107 |
| moles citric acid | 0.0214 | 0.0156 | 0.0075 | 0.0110 | 0.0107 | 0.0107 |
| moles NH₄F | 0.0427 | 0.0213 | 0.0194 | 0.0192 | 0.0213 | 0.0213 |
| mole ratio citrate:calcium | 1.01 | 1.46 | 0.78 | 1.15 | 1.01 | 1.01 |
| mole ratio fluoride:calcium | 2.00 | 2.00 | 2.00 | 2.00 | 2.00 | 2.00 |
| Stable and no precipitate? | YES | YES | YES | YES | YES | NO* |

“YES” indicates that the Example was a stable solution, and no precipitate was observed.
“NO” indicates that a precipitate was observed and therefore the Comparative Example was not a stable solution.
*CEx. A had an equivalent content as EX-1E. However, CEx. A was a Comparative Example because the proper order of addition was not followed, the ingredients were all combined approximately simultaneously. A metastable aqueous solution comprising calcium citrate was not formed first, prior to addition of the fluoride containing compound, and so a precipitate was formed.

TABLE 3
Examples EX-1H, EX-4A, EX-4B, and Comparative Examples CEx. B-CEx. D

| Examples with NH₄F | EX-1H | EX-4A | EX-4B | CEx. B | CEx. C | CEx. D |
|---|---|---|---|---|---|---|
| Components | wt/wt % | wt/wt % | wt/wt % | wt/wt % | wt/wt % | wt/wt % |
| Ca(OH)₂ | 1.19 | 0.78 | 0.78 | 0.78 | 0.71 | 0.78 |
| Citric acid | 2.10 | 1.45 | 1.45 | 1.45 | — | 1.45 |
| maleic acid | • | • | • | — | 2.23 | — |
| NH₄F | 1.18 | 0.61 | 0.58 | 0.018 | 0.71 | — |
| water | 95.53 | 97.16 | 97.18 | 97.78 | 96.34 | 97.77 |
| total | 100.0 | 100.0 | 100.0 | 100.0 | 100.0 | 100.0 |
| moles Ca(OH)₂ | 0.0160 | 0.0105 | 0.0105 | 0.0105 | 0.0096 | 0.0106 |
| moles maleic acid | • | • | • | — | 0.0192 | — |
| moles citric acid | 0.0109 | 0.0075 | 0.0075 | 0.0075 | 0.0000 | 0.0076 |
| moles NH₄F | 0.0319 | 0.0166 | 0.0158 | 0.0005 | 0.0192 | — |
| mole ratio maleate:calcium | • | • | • | — | 2.00 | — |
| mole ratio citrate:calcium | 0.68 | 0.72 | 0.72 | 0.72 | — | 0.72 |
| mole ratio fluoride:calcium | 1.99 | 1.57 | 1.50 | 0.05 | 2.00 | 0.00 |
| Stable and no precipitate? | YES | YES | YES | NO | NO | NO |

TABLE 4A
Examples EX-1F, EX-1G, EX-1J, EX-2A, EX-2B.

| | Examples with NaF | | | | |
|---|---|---|---|---|---|
| | EX-1F | EX-1G | EX-1J | EX-2A | EX-2B |
| Components | wt/wt % | wt/wt % | wt/wt % | wt/wt % | wt/wt % |
| Ca(OH)₂ | 1.19 | 0.73 | 1.17 | 0.78 | 0.78 |
| Citric acid | 2.10 | 2.86 | 2.07 | 1.45 | 1.45 |
| NaF | 1.36 | 0.83 | 2.67 | 0.44 | 0.67 |
| water | 95.35 | 95.57 | 94.08 | 97.37 | 97.10 |
| total | 100.0 | 100.0 | 100.0 | 100.0 | 100.0 |
| moles Ca(OH)₂ | 0.0160 | 0.0099 | 0.0158 | 0.0105 | 0.0105 |
| moles citric acid | 0.0109 | 0.0149 | 0.0108 | 0.0075 | 0.0075 |
| moles NaF | 0.0324 | 0.0198 | 0.0636 | 0.0105 | 0.0160 |
| mole ratio citrate:calcium | 0.68 | 1.50 | 0.68 | 0.72 | 0.72 |
| mole ratio fluoride:calcium | 2.02 | 2.00 | 4.03 | 1.00 | 1.52 |
| Stable and no precipitate? | YES | YES | YES | YES | YES |

TABLE 4B
Examples EX-1K-EX-1N and Comparative Example CEx. E

| | Examples with NaF & NH₄F | | | | |
|---|---|---|---|---|---|
| | EX-1K | EX-1L | EX-1M | EX-1N | CEx. E |
| Components | wt/wt % | wt/wt % | wt/wt % | wt/wt % | wt/wt % |
| Ca(OH)₂ | 3.16 | 0.72 | 2.37 | — | 0.78 |
| Ca(NO3)2•4 H2O | • | — | — | 2.29 | — |
| Citric acid | 8.24 | 1.27 | 6.18 | 2.80 | 1.45 |
| NH₄F | 3.16 | — | 2.37 | • | • |
| NaF | — | 0.82 | — | 0.81 | 0.02 |
| water | 85.44 | 97.20 | 89.08 | 94.08 | 97.70 |
| total | 100.0 | 100.0 | 100.0 | 100.0 | 100.0 |
| moles calcium | 0.0426 | 0.0097 | 0.0320 | 0.0097 | 0.0105 |
| moles citric acid | 0.0429 | 0.0066 | 0.0322 | 0.0146 | 0.0075 |
| moles fluoride | 0.0853 | 0.0196 | 0.0640 | 0.0194 | 0.0005 |
| mole ratio citrate:calcium | 1.01 | 0.68 | 1.01 | 1.51 | 0.72 |
| mole ratio fluoride:calcium | 2.00 | 2.02 | 2.00 | 2.00 | 0.05 |
| Stable and no precipitate? | YES | YES | YES | YES | NO |

TABLE 5
Examples EX-1I, EX-3A, EX-3B, EX-3C and Comparative Example CEx. F.

| | Examples with KF & etidronic•acid | | | | |
|---|---|---|---|---|---|
| | EX-1I | EX-3A | EX-3B | EX-3C | CEx. F |
| Components | wt/wt % | wt/wt % | wt/wt % | wt/wt % | wt/wt % |
| Ca(OH)₂ | 1.19 | 0.78 | 0.78 | 0.074 | 0.78 |
| Citric acid | 2.10 | 1.45 | 1.45 | — | 1.45 |
| Etidronic acid | • | • | — | 0.410 | — |
| KF | 1.86 | 0.61 | 0.92 | 0.115 | 0.03 |
| water | 94.85 | 97.16 | 96.80 | 99.40 | 97.70 |
| total | 100.0 | 100.0 | 100.0 | 100.0 | 100.0 |
| moles Ca(OH)₂ | 0.0160 | 0.0105 | 0.0105 | 0.0010 | 0.0105 |
| moles citric acid | 0.0109 | 0.0075 | 0.0075 | — | 0.0075 |
| moles etidronic acid | • | • | — | 0.0020 | — |
| moles KF | 0.0320 | 0.0106 | 0.0158 | 0.0020 | 0.0005 |
| mole ratio citrate:calcium | 0.68 | 0.72 | 0.72 | — | 0.72 |
| mole ratio etidronic | • | • | — | 2.01 | — |
| acid:calcium | | | | | |
| mole ratio fluoride:calcium | 2.00 | 1.00 | 1.50 | 2.00 | 0.05 |
| Stable and no precipitate | YES | YES | YES | YES | NO |

### Measurement of Free Fluoride in Examples

The free fluoride in various examples was measured on a Mettler Toledo meter with a Cole Parmer fluoride ion selective electrode. The fluoride ion selective electrode was first calibrated with parts per million (ppm) fluoride standards using TISAB II (Total Ionic Strength Adjustment Buffer II, available from Sigma-Aldrich) before measuring Examples for free fluoride content. The example solutions were mixed with 5 ml of TISAB II solution and the ppm concentration of fluoride in these solutions was measured with the fluoride electrode by inserting the electrode into the solution of each example and let it stabilize for 2 minutes before recording the parts per million (ppm) of free fluoride ions in solution.

TABLE 6
Free fluoride measurements of Examples EX-1A, EX-1K, EX-1L

| Examples measured for free F⁻ | EX-1A | EX-1K | EX-1L |
|---|---|---|---|
| mole ratio citrate:calcium | 1.01 | 1.01 | 0.68 |
| mole ratio fluoride:calcium | 2.00 | 2.00 | 2.02 |
| Stable and no precipitate? | YES | YES | YES |
| ppm Free fluoride in solution | 1110 ppm | 1970 ppm | 798 ppm |

### Addition of Phosphate to Stabilized Calcium Example at Various Levels

Table 7 shows that the addition of phosphate at various levels to Example EX-IF does not form a precipitate in the presence of the stabilized complex comprising calcium, citrate, and fluoride.

TABLE 7
Example EX-1F with various levels of phosphate added.

| | EX-1F-P1 | EX-1F-P2 | EX-1F-P3 | EX-1F-P4 | EX-1F-P5 |
|---|---|---|---|---|---|
| Components | wt/wt % | wt/wt % | wt/wt % | wt/wt % | wt/wt % |
| Example EX-1F | 87.5 | 87.5 | 87.5 | 33.3 | 20.0 |
| 1.0 wt/wt % KH₂PO₄ water solution | 12.5 | — | — | — | — |
| 1.0 wt/wt % Na₂HPO₄ water | — | 12.5 | — | — | — |
| solution | | | | | |
| 1.0 wt/wt % Na₃PO₄ water solution | — | — | 12.5 | 66.7 | 80.0 |
| total | 100.0 | 100.0 | 100.0 | 100.0 | 100.0 |
| Stable and no precipitate? | YES | YES | YES | YES | YES |

### Addition of Potassium Nitrate (Dental Desensitizing Agent)

Table 8 shows that the addition of potassium nitrate (KNO₃), a desensitizing agent, to various working examples (Examples EX-IF, EX-1H, and EX-1I) does not form a precipitate in the presence of the stabilized complex comprising calcium, citrate, and fluoride. An amount of 0.25 grams of KNO₃ was added to 5 grams of each of these working examples. The nitrate salt was dissolved, and the solutions remained clear and free of precipitation.

TABLE 8
Examples EX-1F, EX-1H, and EX-1I with added KNO₃.

| | EX-1F-N1 | EX-1H-N1 | EX-1I-N1 |
|---|---|---|---|
| Components | wt/wt % | wt/wt % | wt/wt % |
| EX-1F (containing NaF) | 95.0 | — | — |
| EX-1H (containing NH₄F) | — | 95.0 | — |
| EX-1I (containing KF) | — | — | 95.0 |
| KNO₃ | 5.0 | 5.0 | 5.0 |
| total | 100.0 | 100.0 | 100.0 |
| Nitrate salt dissolved, | YES | YES | YES |
| No precipitation | | | |

### Extended Stability of Examples Comprising Stabilized Calcium Complex Plus Additives

Table 9 shows the extended stability of several examples comprising the stabilized calcium complex of Example EX-1A combined with various additional ingredients useful in dental compositions. In all examples no precipitation was observed even after multiple months (mo.) at room temperature (RT).

TABLE 9
Extended Stability of Example EX-1A with various additional ingredients.

| | EX-1A | FeCl₃ | | | | | |
|---|---|---|---|---|---|---|---|
| | (stock) | (6H₂O) | KH₂PO₄ | arginine | K sorbate | Total | Stability |
| Example | wt/wt % | wt/wt % | wt/wt % | wt/wt % | wt/wt % | wt/wt % | No Precip. |
| EX-1A-1 | 99.5 | 0.5 | — | — | — | 100.0 | >2 mo. RT |
| EX-1A-2 | 99.0 | 1.0 | — | — | — | 100.0 | >2 mo. RT |
| EX-1A-3 | 94.8 | 0.5 | 4.7 | — | — | 100.0 | >2 mo. RT |
| EX-1A-4 | 90.9 | — | — | 9.1 | — | 100.0 | >5 mo. RT |
| EX-1A-5 | 87.0 | — | 8.7 | 4.3 | — | 100.0 | >5 mo. RT |
| EX-1A-6 | 95.3 | — | 4.7 | — | — | 100.0 | >4 mo. RT |
| EX-1A-7 | 94.4 | 0.9 | 4.7 | — | — | 100.0 | >2 mo. RT |
| EX-1A-8 | 99.9 | — | — | — | 0.1 | 100.0 | >5 mo. RT |
| EX-1A-9 | 99.5 | — | — | — | 0.5 | 100.0 | >5 mo. RT |

### Toothpaste Examples

Table 10 shows exemplary toothpaste compositions Examples EX-1F-TP and EX-1J-TP prepared using the stabilized calcium, citrate, fluoride complex Examples EX-1F and EX-1J as stock solutions.

TABLE 10
Toothpaste Examples EX-1F-TP and EX-1J-TP.

| | EX-1F-TP | EX-1J-TP |
|---|---|---|
| Components | wt/wt % | wt/wt % |
| EX-1F (Stock Solution) | 40.96 | — |
| EX-1J (Stock Solution) | — | 40.95 |
| Cellulose gum | 0.71 | 0.72 |
| glycerin | 8.14 | 8.19 |
| sorbitol | 26.55 | 26.52 |
| spearmint flavor | 1.01 | 1.02 |
| sodium lauryl sulfate | 0.80 | 0.82 |
| TiO₂ | 0.73 | 0.72 |
| sodium saccharin | 0.27 | 0.28 |
| ZEODENT 165 silica | 6.60 | 6.61 |
| SIDENT 9 silica | 14.22 | 14.17 |
| Total | 100.0 | 100.0 |
| Stable and no precipitate? | YES | YES |

### Mouthwash Example

Table 11 shows an exemplary mouthwash formulation EX-1F-MW which was prepared using stabilized calcium, citrate, fluoride complex Example EX-1F as a stock solution.

TABLE 11
Mouthwash Example EX-1F-MW

| Components | wt/wt % |
|---|---|
| EX-1F (stock solution) | 82.99 |
| hydroxy ethyl cellulose | 0.10 |
| glycerin | 15.77 |
| sodium benzoate | 0.11 |
| Na citrate | 0.88 |
| sucralose | 0.15 |
| total | 100.0 |
| Stable and no precipitate? | YES |

### Stock Solutions for Dental Compositions

Table 12 shows exemplary stock solutions for subsequent dental compositions prepared using Examples EX-1A, EX-1B, and EX-1M as stock solutions.

TABLE 12
Stock Solutions for Dental Compositions

| | EX-1A- | EX-1B- | EX-1A- | EX-1M- |
|---|---|---|---|---|
| | SS-1 | SS-2 | SS-3 | SS-4 |
| Components | wt/wt % | wt/wt % | wt/wt % | wt/wt % |
| EX-1A | 90.0 | — | 77.0 | — |
| EX-1B | — | 77.0 | — | — |
| EX-1M | — | — | — | 77.0 |
| tartaric acid | 10.0 | — | — | — |
| K₂SO₄ | — | 5.4 | 5.4 | 5.4 |
| KH₂PO₄ | — | 17.6 | 17.6 | 17.6 |
| Total | 100.0 | 100.0 | 100.0 | 100.0 |
| Stable and no | YES | YES | YES | YES |
| precipitate? | | | | |
| Extended | >3 mo. | >3 mo. | >3 mo. | >3 mo. |
| Stability | RT | RT | RT | RT |

### “Paste B” Preparations for Resin-Modified Glass Ionomer (RMGI) Dental Compositions

Resin-Modified Glass Ionomer (RMGI) cements are two-part systems that when combined harden (cure). The RMGI examples below were prepared as paste-paste, (Paste “A” and Paste “B”) two-part reactive systems. The calcium stabilized complex examples (EX-1A, EX-1B, and EX-1M) described above were used in Stock Solutions described in Table 12. The Stock Solutions described in Table 12 were then used in the preparation of the Paste “B” Examples shown in Table 13, below.

TABLE 13
Paste “B” portion of RMGI Cement Two-Part System

| | RMGI Paste “B” | | |
|---|---|---|---|
| | EX-Paste-B1 | EX-Paste-B2 | EX-Paste-B3 |
| Components | wt/wt % | wt/wt % | wt/wt % |
| EX-1B-SS-2 | 11.3 | — | — |
| EX-1A-SS-3 | — | 11.3 | — |
| EX-1M-SS-4 | — | — | 11.3 |
| K₂S₂O₈ | 1.94 | 1.94 | 1.94 |
| BHT | 0.13 | 0.13 | 0.13 |
| CPQ | 0.1 | 0.1 | 0.1 |
| HEMA | 20.1 | 20.1 | 20.1 |
| GDMA | 4.99 | 4.99 | 4.99 |
| VBCP | 23.87 | 23.87 | 23.87 |
| silca/zirconia filler | 37.36 | 37.36 | 37.36 |
| fumed silica | 0.21 | 0.21 | 0.21 |
| total | 100 | 100 | 100 |

### Preparation of Resin-Modified Glass Ionomer (RMGI) Cements

To prepare a RMGI using the prepared Paste “B” Examples shown in Table 13, a suitable Paste “A” was needed to react with Paste “B.” A source of Paste “A” was obtained by removing/using only the white-colored paste (the base paste) from one of the two syringe barrels of the 3M commercially available product Rely X™ Luting Plus Automix Resin-Modified Glass Ionomer Cement. Alternatively, the white-colored Paste “A” (the base paste) may also be obtained from commercially available 3M product RelyX™ Luting Plus Cement Clicker™ Dispenser Refill. Paste “A” and Paste “B” were mixed by hand for 20 seconds in a 5:4 part ratio (A:B) respectively, on a mixing pad using a dental spatula. The mixed examples were then placed in a 37° C. oven to cure and harden (set). The mixtures were tapped with the spatula to verify they were set, the time was recorded.

The set times of these RMGI examples were compared to a control sample which was prepared in the same manner but using commercially available Rely X™ Luting Plus Automix Resin-Modified Glass Ionomer Cement, which comes in a double barrel syringe systems already containing a Paste “A” and Paste “B.” Table 14 shows the set times (minutes: seconds) for the Resin-Modified Glass Ionomers Examples EX-RMGI-1, EX-RMGI-2, and EX-RMGI-3, which comprised as source material, Examples EX-1A, EX-1B, and EX-1M, respectively.

TABLE 14
Set Times for Resin-Modified Glass Ionomers Examples.

| Components | EX-RMGI-1 | EX-RMGI-2 | EX-RMGI-3 | Control RMGI |
|---|---|---|---|---|
| Paste “A” | Paste A from | Paste A from | Paste A from | Paste A from |
| | RelyX ™ | RelyX ™ | RelyX ™ | RelyX ™ |
| | Luting Plus | Luting Plus | Luting Plus | Luting Plus |
| Paste “B” | EX-Paste-B1 | EX-Paste-B2 | EX-Paste-B3 | Paste B from |
| | | | | RelyX ™ |
| | | | | Luting Plus |
| Set time | 3:00 | 3:10 | 3:10 | 2:50 |
| min:sec | | | | |

### Extended Stability Testing of Resin-Modified Glass Ionomer (RMGI) Cements

Commercially available 3M product RelyX™ Luting Plus Cement Clicker™ Dispensers were used to provide product simulated storage containers for an experiment to evaluate the extended stability at elevated temperature of RMGI cement Examples made using Examples EX-1A, EX-1B, and EX-IM. The yellow-colored catalyst material (commercial Paste “B”) was removed from the respective syringe cylinders of the Rely X™ Luting Plus Cement Clicker™ Dispenser product. The emptied syringe cylinders were then filled with the experimental Examples EX-Paste-B1, EX-Paste-B2, and EX-Paste-B3. For each dispenser the original commercial Paste “A” was left in the adjacent syringe cylinder. These simulated products containing the experimental Examples EX-Paste-B1, EX-Paste-B2, and EX-Paste-B3 were stored for multiple weeks in a temperature-controlled oven at 45° C. Table 15 shows the set times (minutes: seconds) for the experimental Resin-Modified Glass Ionomers Examples EX-RMGI-1, EX-RMGI-2, and EX-RMGI-3, (prepared using EX-1A, EX-1B, and EX-IM, respectively), after extended storage in a product container at 45° C. for 4 weeks, 6 weeks, 10 weeks.

TABLE 15
Extended Stability Set Times for Resin-
Modified Glass Ionomers Examples.

| | EX-RMGI-1 | EX-RMGI-2 | EX-RMGI-3 |
|---|---|---|---|
| Storage time at | Set time | Set time | Set time |
| 45° C. | (min:sec) | (min:sec) | (min:sec) |
| Time 0, initial | 3:00 | 3:10 | 3:10 |
| 4 weeks | 3:10 | 3:10 | 3:15 |
| 6 weeks | 3:10 | 3:15 | 3:20 |
| 10 weeks | 3:30 | 3:30 | 3:30 |

### Example Including Curable Monomers and Copolymer for Dental Compositions

### Example EX-5A

Example EX-5A was prepared in the following manner. An amount of 0.5 grams of Example EX-1B shown in Table 2 (citrate: calcium mole ratio of 1.5:1 and fluoride: calcium mole ratio of 2:1) was mixed with 0.5 g of 10% VBCP solution in water. An amount of 1.5 g of HEMA was added to the mixture. The HEMA had been initiated with 0.22% CPQ, 1.0% EDMAB, 0.3% DPIHFP, and 0.1% 9,10-EDMOA. Next, an amount of 0.5 g of CDMA/GDMA oligomer blend was added to the mixture. The CDMA/GDMA oligomer blend had been initiated with 0.22% CPQ, 1.0% EDMAB, 0.3% DPIHFP, and 0.1% 9,10-EDMOA. Finally, an amount of 0.1 g of SR454 was added to the mixture. The SR454 had been initiated with 0.22% CPQ, 1.0% EDMAB, 0.3% DPIHFP, and 0.1% 9,10-EDMOA. This mixture formed a clear solution that appeared to be stable, no precipitation.

### Additional Comparative Examples: CEx.G-CEx.K

### Comparative Example CEx.G

A 0.1M aqueous solution of calcium nitrate was mixed with a separate 0.2M aqueous solution of ammonium fluoride. Upon mixing these two solutions, an insoluble calcium fluoride salt precipitate was formed (No chelating agent (e.g., citrate) present).

### Comparative Example CEx.H

An amount of 0.32 grams calcium succinate was added to 9.68 g of DI water (3.2% wt/wt). At this concentration calcium succinate was found to be only sparingly soluble. An amount of 10 grams of 0.4 M NaF solution was added to the calcium succinate mixture. The new mixture did not form clear solution (was not fully dissolved); instead, a cloudy slurry (precipitate) was formed (No adequate chelating agent (e.g., citrate) present).

### Comparative Example CEx.I

An amount of 0.31 g calcium fumarate was added to 9.72 g of DI water (3.1% wt/wt). At this concentration calcium fumarate was found to be only sparingly soluble. An amount of 10 grams of 0.4 M NaF solution was added to the calcium fumarate mixture. The new mixture did not form clear solution (was not fully dissolved); instead, a cloudy slurry (precipitate) was formed (No adequate chelating agent (e.g. citrate) present).

### Comparative Example CEx.J

A solution of Ca (OH) 2 and Citric acid was prepared with molar ratios of 0.1 moles hydroxide to 0.15 moles citrate. Initially a clear solution formed. However, after waiting several (approximately 30) minutes, a precipitate was formed, presumably calcium citrate or its hydrates. Thus, this mixture was not able to react effectively with a fluoride salt at this point. This is a Comparative Example because the proper order of addition was not followed, in that the fluoride containing compound was not added within 5 minutes of the formation of the metastable aqueous solution comprising calcium citrate (i.e., prior to the precipitation of the calcium citrate or its hydrates).

### Comparative Example CEx.K

An amount of 2.282 grams of calcium citrate tetrahydrate was stirred in 18.20 grams of DI water (11.1% wt/wt). The calcium citrate tetrahydrate was essentially insoluble at this concentration. An amount of 20.07 grams of 0.4M ammonium fluoride solution was added to the calcium citrate tetrahydrate mixture. No change in the mixture was visually noticed, calcium citrate tetrahydrate remained undissolved, not in solution.

1. An aqueous oral care composition comprising:
a complex comprising:
calcium,
fluoride, and
citrate; and
water;
wherein the aqueous oral care composition comprises at least 2 moles of citrate for every 3 moles of calcium;
wherein the aqueous oral care composition comprises at least 1 mole of fluoride for every 1 mole of calcium;
wherein water is present in an amount of at least 50 wt %, based on the total weight of the aqueous oral care composition; and
wherein the aqueous oral care composition is free of precipitates.

2. (canceled)

3. The aqueous oral care composition of claim 1, characterized by pH between 2 and 11.

4. The aqueous oral care composition of claim 1, further comprising one or more of: free fluoride, a calcium citrate complex, and calcium fluoride nanoparticles.

5. The aqueous oral care composition of claim 1, wherein calcium is present in an amount of at least 20 ppm, based on the total weight of the aqueous oral care composition.

6. The aqueous oral care composition of claim 1, wherein fluoride is present in an amount of at least 20 ppm, based on the total weight of the aqueous oral care composition.

7. The aqueous oral care composition of claim 1, further comprising free fluoride is-present in the composition in an amount of at least 50 ppm based on the total weight of the aqueous oral care composition.

8. The aqueous oral care composition of claim 1, further comprising a water-soluble phosphate salt.

9. The aqueous oral care composition of claim 1, further comprising a desensitizing agent, the desensitizing agent comprising a water-soluble potassium salt.

10. A method of making an aqueous oral care composition of claim 1, the method comprising:
reacting in aqueous solution a calcium source with a citrate source to form a metastable aqueous solution comprising calcium citrate or its hydrates;
adding a water-soluble fluoride salt solution to the metastable aqueous solution prior to precipitation of the calcium citrate or its hydrates.

11. The method of claim 10, wherein the calcium source comprises calcium hydroxide, calcium acetate, calcium carbonate, or calcium hydrogen carbonate.

12. The method of claim 10, wherein the citrate source comprises citric acid.

13. The method of claim 10, wherein the calcium source is present in the metastable solution in an amount of at least 5 ppm, based on the total weight of the metastable solution.

14. The method of claim 10, wherein the water-soluble fluoride solution comprises a water-soluble fluoride salt and water, and wherein the water-soluble fluoride salt comprises sodium fluoride, potassium fluoride, silver diamine fluoride, or ammonium fluoride.

15. The method of claim 14, wherein the water-soluble fluoride salt is present in the water-soluble fluoride salt solution in an amount of at least 20 ppm, based on the total weight of the water-soluble fluoride salt solution.

16. (canceled)

17. The method of claim 10, wherein the adding of the water-soluble fluoride solution to the metastable solution is within 2 minutes or less from formation of the metastable solution.

18. (canceled)

19. A method of providing fluoride to a patient's tooth surface, the method comprising:
applying the aqueous oral care composition of claim 1, to the patient's tooth surface.

20. The method of claim 19, wherein prior to applying the aqueous oral care composition, the aqueous oral care composition is incorporated into another composition.

21. A toothpaste composition comprising:
the aqueous oral care composition of claim 1.
